(19) United States
(12) Patent Application Publication
(10) Pub. No.: US 2009/0005767 A1
(43) Pub. Date: Jan. 1, 2009

(54) DEVICE AND METHOD FOR BENIGN PROSTATIC HYPERPLASIA LASER TREATMENT

(75) Inventors: KELLY B. MORAN, Wilbraham, MA (US); David S. Turk, Broadview Heights, OH (US)

Correspondence Address: MCCARTER & ENGLISH LLP;CITYPLACE I, 185 ASYLUM STREET, HARTFORD, CT, 06103, US

(73) Assignee: CeramOptec Industries Inc.

(21) Appl. No.: 12/123,114

(22) Filed: May 19, 2008

Related U.S. Application Data
(60) Provisional application No. 60/930,562, filed on May 17, 2007.

(51) Int. Cl. A61B 18/18

(52) U.S. Cl. 606/10

(57) ABSTRACT

A method and device is provided for high power BPH laser treatments in a doctor's office or clinic setting wherein only local anesthetics are employed, making the treatments administrable in a doctor's office or a clinic, and allowing the patient to remain conscious throughout the procedure so that the doctor can interact with the patient while administering the treatment. The BPH treatments can be administered in a single short duration, i.e., each session lasting for not more than about 45 minutes and having a lasing time within the range of about 10 minutes to about 20 minutes, thus limiting the amount of discomfort and anxiety a patient might experience. The relatively high power treatment coupled with the relatively short treatment time allows doctors to assess the results of the treatment session within minutes after completing the procedure. The high power BPH laser treatments are ablative in nature wherein laser radiation is provided, generally in a pulsed mode, at preselected power output levels of at least about 100 Watts at wavelengths of 980 m and/or 1460 nm. The device includes a stable high power laser source and an optical fiber optically connected to the laser source that is capable of carrying high power laser radiation. The device is mobile and/or portable and can be operated with a standard 110/220 volt alternating current (AC) power source.

### CROSS-REFERENCE TO PRIORITY APPLICATION

This patent application claims priority under 35 U.S.C. § 119 to co-pending U.S. provisional patent application Ser. No. 60/930,562, filed May 17, 2007, entitled “Device And Method For Benign Prostatic Hyperplasia Laser Treatment”, which is hereby expressly incorporated by reference as part of the present disclosure.

### BACKGROUND OF THE INVENTION

1. Field of the Invention

The present invention relates to medical laser treatments, and more particularly, to techniques for benign prostatic hyperplasia (“BPH”) laser treatments in a doctor's office.

2. Invention Disclosure Statement

Benign prostatic hyperplasia (“BPH”) or “enlarged prostate” refers to non-cancerous (benign) growth of the prostate gland. BPH is sometimes referred to as soft tissue in the urinary tract. BPH is the most common prostate problem in men over 50 years of age. BPH begins with the formation of microscopic nodules typically around 25 years of age but rarely produces symptoms before a man reaches age 40. An estimated 6.3 million men in the United States have BPH and the disease is responsible for 6.4 million doctor visits and more than 400,000 hospitalizations per year.

The exact cause of BPH is unknown but is generally thought to involve hormonal changes associated with the aging process. For example, testosterone likely plays a role in promoting BPH. Testosterone is continually produced throughout a man's lifetime and is a precursor to dihydrotestosterone (“DHT”), which induces rapid growth of the prostate gland during puberty and early adulthood. When fully developed, the prostate gland is approximately the size of a walnut and remains this size until a man reaches his mid-forties. At this point, the prostate begins a second period of growth, which for many men leads to BPH later in life.

In contrast to the overall enlargement of the prostate gland during puberty and early adulthood, benign prostate growth occurs only in a central area of the prostate gland called the transition zone, which wraps around the urethra. As this area of the prostate gland grows, the prostate gland presses and constricts the urethra, potentially causing a number of lower urinary tract symptoms (“LUTS”), such as difficult urination (obstructive symptoms) and painful urination (storage symptoms). The effects of LUTS can, over time, impact other related organs, such as the bladder. For example, the bladder can become weakened and lose the ability to empty itself.

Obstructive symptoms such as intermittent flow or hesitancy before urinating can severely reduce the volume of urine being eliminated from the body, a condition referred to as acute urine retention. If left untreated, acute urine retention can lead to other serious complications such as bladder stones, urinary tract infections, incontinence, and in rare cases, bladder damage and kidney damage. These complications are more prevalent in older men who are also taking anti-arrhythmic drugs or anti-hypertensive (non-diuretic) medications. Further, in addition to the physical problems associated with BPH, many men also experience anxiety and a reduced quality of life.

BPH symptoms can be treated through the removal of prostatic tissue using laser treatments, some of which can be performed in a doctor's office. For example, treatments using the Indigo® Laser system (manufactured by Ethicon Endo-surgery, Inc. a division of Johnson & Johnson) can be performed in a doctor's office or outpatient surgery center typically using local or general anesthesia. The treatments performed in the doctors' office generally function by using thermal damage to destroy excess prostate tissue, employing different wavelengths of electromagnetic radiation from visible (Indigo) to microwave and through radio frequency wavelengths. The tissue is then sloughed off over time, by action of the body's own natural repair process. These BPH treatments performed in the doctors' office are, thus, limited to the removal of only small amounts of tissue, i.e., on the order of about 25 grams or so of tissue. Further, according to this procedure, the effects of a particular treatment session can be truly assessed only after about 6-8 weeks. The determination that further treatment is needed entails additional 6-8 week period(s). This is a long treatment period that can be difficult for patients to endure. With current BPH in-office, minimum invasive treatment technology, including laser treatment technology, only about a 60 percent efficacy is encountered.

Currently, only the extended-visit BPH treatment techniques described above can be performed in a doctor's office, which really limits the number of procedures that can be performed without admitting the patient into a hospital. For example, treatments, wherein 50 grams or more of prostatic tissue are to be removed, require significant extra time or dosage and thus need to be performed in a normal operating room using general anesthesia, and therefore must be performed in a hospital. There are serious risks associated with the use of a general anesthetic. Further, since a patient under general anesthesia is rendered unconscious, doctors are unable to communicate with the patient during the procedure, e.g., so as to help minimize the occurrence of over-treatment and postoperative pain/discomfort.

Thus, currently, patients can either choose to undergo long-term BPH treatments in their doctor's office (which can, as described above, take longer than 6-8 weeks to ascertain cure) or take the risk of general anesthesia and have the procedure performed in a hospital. Both options have serious drawbacks. Photoselective vaporization of the prostate (U.S. Pat. No. 6,986,764 to Davenport et al.) involves transmitting laser radiations with specific average irradiance in the treatment area to form a spot of preset size. The '764 patent shows a high-power potassium-titanyl-phosphate (“KTP”) laser, also called the “greenlight” laser. The delivered laser radiation has a wavelength between 200 nm and 650 nm, and has an average irradiance in the treatment area greater than about 10 kilowatts/cm², in a spot size of at least 0.05 mm². The use of high-power potassium-titanyl-phosphate involves the disadvantages attributed to irradiating a prostate with greenlight laser radiation.

As such, there remains a need for an improved BPH laser treatment device and method for the effective and efficient alleviation of BPH symptoms, that can be performed in a doctor's office or in a true outpatient setting, i.e., that does not require general anesthetics, can remove the required amount of prostatic tissue, and produces results that can be evaluated in a relatively short time period. The present invention is directed towards this need.

### OBJECTIVES AND BRIEF SUMMARY OF THE INVENTION

It is an objective of the present invention to be able to perform a BPH laser treatment method in a doctor's office and/or in an outpatient setting, using only a local anesthetic in minimal quantities.

It is another objective of currently preferred embodiments of the present invention to use the BPH laser treatment method to remove amounts of prostatic tissue that exceed 50 grams, e.g., up to about 100 to about 200 grams of prostatic tissue. It is still another objective of currently preferred embodiments of the present invention to have short treatment times, e.g., each treatment session lasting for not more than about 45 minutes and having a lasing time of about 10 minutes to about 20 minutes.

It is yet another objective of the currently preferred embodiments of the present invention to provide a BPH laser treatment device and system that is portable. It is a further objective of the present invention to provide a BPH laser treatment method that is ablative in nature.

It is still a further objective of the currently preferred embodiments of the present invention to make the medical laser device portable and capable of operating on a standard 110/220 volt power source.

Briefly stated, the present invention provides a method and device for high power BPH laser treatments in a doctor's office or clinic setting wherein only local anesthetics are employed, making the treatments administrable in the doctor's office or clinic, and allowing the patient to remain conscious throughout the procedure so that the doctor can interact with the patient while administering the treatment and thereby eliminate potentially serious complications associated with general anesthetics used in conjunction with hospital-administered treatments. The currently preferred embodiments of the BPH treatment can be administered in a single short duration, i.e., each session lasting for not more than about 45 minutes and having a lasing time within the range of about 10 minutes to about 20 minutes, thus limiting the amount of discomfort and anxiety a patient might experience. Further, a high power treatment coupled with a short treatment time allows doctors to assess the results of a treatment session within minutes after completing the procedure, as compared to conventional procedures that can require many weeks before the results can be evaluated. According to the present teachings, the high power BPH laser treatments are ablative in nature wherein laser radiation is provided, generally in a pulsed mode, at preselected power output levels of at least about 100 Watts at wavelengths of about 980 nm and/or about 1460 nm. The device for performing the BPH treatments includes a stable high power laser source and an optical fiber optically connected to the laser source. The optical fiber is capable of carrying high power laser radiation. The device is mobile and/or portable and can be operated with a standard 110 and/or 220 volt alternating current (AC) power source.

The above and other objects, features and advantages of the present invention and of the currently preferred embodiments thereof will become apparent from the following description read in conjunction with the accompanying drawings.

### BRIEF DESCRIPTION OF THE DRAWINGS

FIG. 1 is a diagram illustrating an exemplary laser treatment for treating benign prostatic hyperplasia (“BPH”).

FIG. 2 is a diagram illustrating exemplary techniques used to irradiate and ablate at all areas of a treatment site.

FIG. 3 is a diagram illustrating an exemplary BPH laser treatment device for performing the BPH laser treatment of FIG. 1 in a doctor's office or clinic.

FIG. 4A is a diagram illustrating an optical fiber having distally a side-firing tip.

FIG. 4B is a diagram illustrating an optical fiber having distally a bent tip.

FIG. 4C is a diagram illustrating an optical fiber having a straight tip.

### DETAILED DESCRIPTION OF PREFERRED EMBODIMENTS

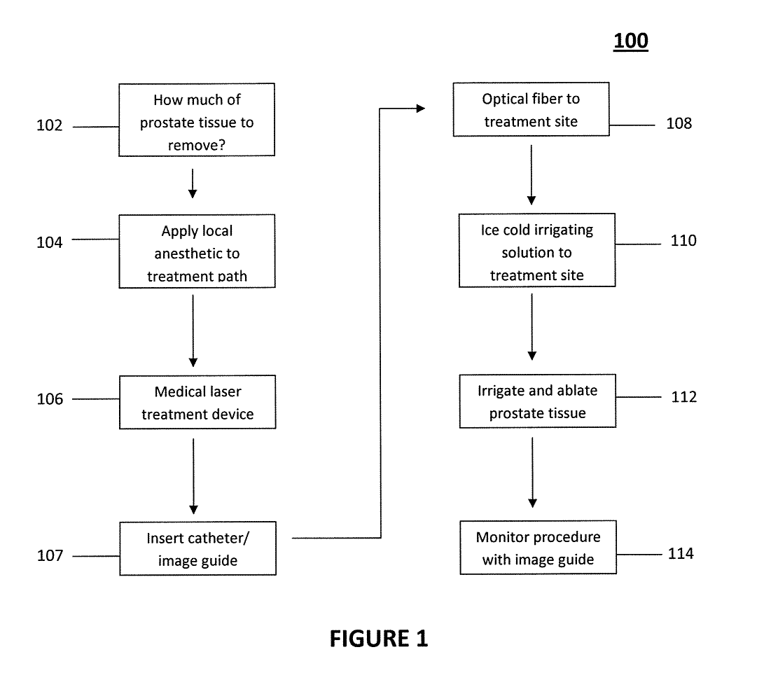

FIG. 1 is a diagram illustrating exemplary laser treatment 100 for treating benign prostatic hyperplasia (“BPH”). BPH laser treatment 100 is a high power laser treatment that advantageously requires only a local anesthetic and thus can be performed on a patient while the patient is in a doctor's office or a clinic. Further, being a high power laser treatment, BPH laser treatment 100 involves short treatment sessions, typically lasting only up to about 45 minutes. The lasing time of prostatic tissue can be anywhere between about 10 minutes and about 20 minutes depending upon the size of the prostatic tissue being ablated.

In step 102, a determination is made as to how much of the patient's prostate tissue has to be removed to effectively treat the BPH symptoms. To make this determination, conventional imaging techniques can be used. For example, transrectal ultrasound (“TRUS”) techniques can be employed to obtain a computer-generated image of the prostate. From that image, doctors can estimate the amount of excess, i.e., hyperplastic, prostate tissue present that has to be removed. Based on the determination of how much of the patient's prostate to remove, a treatment power level can be selected (see description of step 112, below). According to the present teachings, amounts of prostate tissue in excess of 50 grams can be removed in a single treatment session. It is in this step that the doctor predetermines (pre-selects) a treatment site, i.e., a portion of the prostate to which laser treatment will be administered, as described below. Predetermining the treatment site location allows for positioning of the laser treatment device and associated components (e.g., imaging guides), as is described in detail below.

In step 104, a local anesthetic is applied along the patient's urinary canal, i.e., urethra. According to the present teachings, the urethra acts as a treatment path, i.e., providing treatment access to the prostate. In an exemplary embodiment, the local anesthetic used is a gel containing Lidocaine or a saline solution of Lidocaine. Lidocaine is a common local anesthetic that is marketed, for example, under the brand names Xylocaine® and Xylocard® by Abraxis Pharmaceutical Products of East Schaumburg, Ill., USA. The local anesthetic is applied in minimal quantities, e.g., so as to sufficiently cover the treatment path, but not so as to necessarily enter the prostate.

Any suitable method of applying the local anesthetic to the treatment path can be employed. For example, a catheter can be inserted into the urethra and used to apply the saline solution of Lidocaine along the treatment path. Once the local anesthetic is applied, a short waiting period of 10-20 minutes is needed before the lasing portion of the treatment can be commenced, i.e., to allow for the anesthetic to take effect, reducing sensation. According to an alternative embodiment, a local anesthetic, such as a penile block anesthetic, can be administered to the treatment path via injection delivery means. Optionally, an antibacterial agent(s) can also be administered to the treatment site/along the treatment path to prevent infection. Suitable antibacterial agents include, but are not limited to, broad spectrum antibiotic solutions. According to an exemplary embodiment, the antibacterial agent is administered via injection delivery means to the treatment site and/or along the treatment path. In an alternative embodiment, the local anesthetic can be employed using a triple block of (1) transrectal ultrasound and injection of Lidocaine into the periprostatic space, (2) insertion of Lidocaine jelly into the urethra, and (3) intraprostatic injection of Lidocaine.

Since only a local anesthetic is used, the patient remains conscious throughout the entire treatment. This has several notable advantages. First, the dangers associated with the use of a general anesthetic, such as complications arising from interactions from the anesthesia drugs which can lead to illness or even death, are avoided. Second, the doctor can interact with the patient while administering the treatment. The patient can thus tell the doctor if pain is present, which can indicate over-treatment, or other problems. As such, postoperative pain and discomfort are minimized by the present invention. In most cases, the patient is fully functional and able to leave the doctor's office or clinic shortly after the treatment is completed.

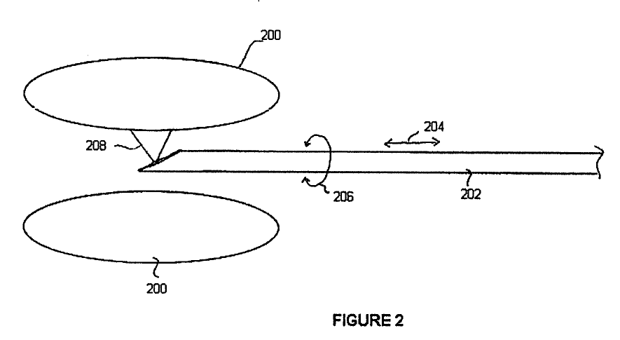

In step 106, a medical laser treatment device is provided. As will be described, for example, in conjunction with the description of FIG. 2, below, the medical laser treatment device includes a high power laser radiation source optically coupled to an optical fiber. The optical fiber is capable of handling high laser power and distributing it obliquely/perpendicularly to a longitudinal axis of the fiber.

In step 107, prior to beginning the lasing phase of the treatment, a scope containing an image guide can be inserted along the treatment path to just short of the predetermined treatment site. The image guide will provide the doctor with a real-time image of the treatment, as is described in detail below. The term “scope” as used herein includes but is not limited to flexible and rigid endoscopes, systoscopes, etc. which can be used for direct visualization of internal structures. To begin the lasing phase of the treatment, in step 108, a distal end of the optical fiber is inserted into the urethra, through the scope (see step 107, described above) and along the treatment path until the distal end of the fiber is adjacent to the prostate gland treatment site. In step 110, an ice cold irrigating solution is provided to the treatment site. According to an exemplary embodiment, the irrigating solution comprises a 0.9 percent saline solution that is introduced to the treatment site having a temperature of between about zero degrees Celsius (° C.) and about 5° C. As will be described in detail below, the medical laser treatment device can include means for delivering/removing the ice-cold irrigating solution to/from the treatment site. In alternative embodiments, the ice-cold irrigating solution can comprise water. While water is a viable alternative to a saline solution, to remain in a liquid form it must be introduced to the treatment site at a temperature above 0° C., e.g., from about 2° C. to about 5° C.

In step 112, the medical laser treatment device is used to irradiate and ablate the prostate tissue at the treatment site using laser radiation produced at a preselected power level and wavelength. According to an exemplary embodiment, the power level is preselected to be at least 100 Watts, depending on primarily the amount of tissue which needs to be removed and the wavelength is either 980 nm or 1,460 nm, or both. Typically the ablative effect is greater but more confined with the longer wavelength (1,460 nm), while the shorter wavelength (980 nm) generally has photocoagulation as well as thermal coagulation. Thus, the wavelength(s) can be chosen depending on the desired results.

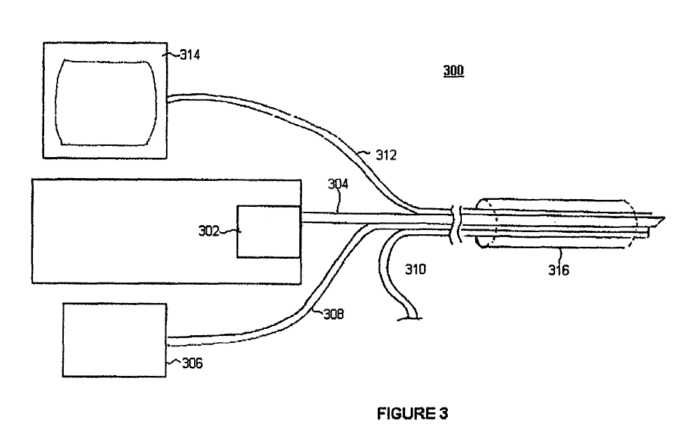

In step 114, the irradiation and ablation is monitored with an image means, such as an image guide, present at the treatment site. An exemplary image means is shown, for example in FIG. 3, described below. Doctors can monitor the progress of the treatment in real time and avoid removing more prostate tissue than is necessary. Monitoring the procedure with an image guide is greatly aided by a significantly less or reduced amount of blood, around the treatment field. In the present invention the use of the 980±20 nm and/or 1460±60 nm achieves a substantively reduced amount blood around the treatment field at appropriate power levels, as described in detail below.

BPH laser treatment 100 takes on average between about 10 minutes and about 20 minutes of lasing time while the treatment session can last for approximately 45 minutes. In some embodiments of the present invention, the BPH laser treatment is less than about an hour, is preferably less than about 50 minutes, and is most preferably about 45 minutes or less, or within the range of about 30 to 45 minutes, depending on the amount of prostatic tissue to be removed. This short treatment duration helps to minimize any amount of discomfort or anxiety the patient may experience during the treatment. Further, as compared to conventional BPH treatments which involve lengthy treatment sessions, including patient recovery time from anesthesia, with BPH laser treatment 100 a doctor is able to treat as many patients in a less time duration. The present invention with its high power treatment coupled with a short treatment time further allows doctors to assess the results of a treatment session within minutes after completing the procedure, as compared to conventional procedures that can require many weeks before the results can be evaluated.

According to an exemplary embodiment, BPH laser treatment 100 produces significantly less blood around the treatment site. A substantially reduced blood field permits optical means, mentioned in step 114 and as will be described in detail below, to be employed during the treatment to allow doctors to monitor the treatment in real time. As a result, safer, more accurate tissue removal can be achieved. If bleeding occurs during the treatment, the power level can be increased to stop the bleeding. By way of example only, if a treatment being performed at 100 Watts results in some bleeding, the power level can be increased, for example, to 120 Watts, so as to stop the bleeding.

One advantage of the wavelengths used in the currently preferred embodiments of the present invention (i.e., about 980 nm±20 nm and/or about 1460 nm±60 nm) is that the 980 nm radiation is about equally absorbed in blood and water, thus coagulating the blood in the prostate and preventing bleeding at the treatment site and rapidly ablating the prostate tissue (which is nearly about 90% water) at high power levels (i.e., preferably greater than about 80 Watts, and most preferably about 100 Watts or greater). Similarly, the 1460 nm radiation is highly absorbed in water and likewise rapidly ablates the prostate tissue. As a general matter, the higher the power, the more rapid is the absorption by and coagulation of the blood, and the more rapid is the ablation of the prostatic tissue. Also as a general matter, the higher the power, the lesser is the time that the laser needs to be on during the procedure to ablate a given amount of prostate tissue, and the lesser is the amount of residual heat absorbed by the prostate tissue surrounding the treatment site. As a result, a significant advantage of the method and device of the present invention is that they can be employed to rapidly ablate significant volumes of prostate tissue in a bloodless or substantially bloodless treatment field, and furthermore, enable such treatments, including treatments involving the ablation of significant amounts of prostate tissue (e.g., at least about 35 to 40 grams, and if desired, greater than 50 grams and including up to about 100 to about 200 grams) to be performed under local anesthetic in a doctor's office or like outpatient or clinical setting in a relatively short period of time (e.g., less than about an hour, preferably less than about 50 minutes, and most preferably about 45 minutes or less) with lesser discomfort in comparison to prior art procedures.

Although the medical laser treatment device can be operating in either a pulsed or continuous mode, in the currently preferred embodiments the device is preferably operated in a pulsed mode, wherein the laser radiation is pulsed on for a certain duration and then off for a certain other duration. The pulsed mode, either on or off, can be achieved through use of a gated means, i.e., such that the laser radiation is pulsed off by stopping the radiation path, e.g., as opposed to turning off the laser. One exemplary pulse sequence that can be employed is to have the laser radiation pulsed on for a duration of up to about 0.2 second and pulsed off for a duration of up to about 0.01 second. Another exemplary pulse sequence that can be employed is to have the laser radiation pulsed on for a duration of up to about 0.1 second and pulsed off for a duration of up to about 0.01 second.

As shown in FIG. 2, to irradiate and ablate at all areas of treatment site 200, optical fiber 202 can be moved back and forth along the treatment path (as indicated by arrow 204) and/or twisted (as indicated by arrow 206) during the treatment. As described above, laser power 208 is distributed obliquely/perpendicularly to a longitudinal axis of optical fiber 202.

FIG. 3 is a diagram illustrating exemplary BPH laser treatment device 300 for performing BPH laser treatment 100 in a doctor's office or clinic. Device 300 includes high wattage laser source 302, capable of outputting at least 100 Watts of laser radiation, and optical fiber 304 optically coupled to laser source 302. Optical fiber 304 is capable of handling the high wattage output from laser source 302. According to an exemplary embodiment, optical fiber 304 is a glass core/cladding optical fiber. Medical laser treatment device 300 emits light typically at a wavelength of about 980±20 nm or about 1,460±60 nm. The device can also be set up to have both wavelengths available.

Device 300 is a mobile device, in that it can be transported from one doctor's office, or one clinic, to another to perform BPH laser treatments. According to one exemplary embodiment, device 300 is portable. Device 300 can operate on a standard 110/220 volt power source, thus adding to the device's versatility and mobility/portability.

As described above, according to step 110 of BPH laser treatment 100, an ice cold irrigating solution is provided to the treatment site. Thus, device 300 may also include irrigating solution reservoir 306 into which ice cold irrigating solution can be placed. Alternatively, reservoir 306 may itself comprise a cooling unit (not shown) adapted to cool the irrigating solution in reservoir 306 to the appropriate temperature, thus eliminating the need for an external source of ice cold irrigating solution and further adding to the device's versatility and mobility/portability.

A means for delivering/removing the irrigating solution to/from the treatment site can be provided by way of one or more fluid channels fluidly connected to reservoir 306. By way of example only, fluid channel 308 (fluidly connected to reservoir 306) can run at least a portion of the way along optical fiber 304, terminating proximate to the distal end of fiber 304, so as to deliver the ice cold irrigating solution from reservoir 306 to the treatment site. A second fluid channel, i.e., fluid channel 310, can also be present to remove irrigating solution from the treatment site and deliver it to a separate receptacle (not shown) for disposal.

As described above, an image guide may be used to provide real-time images during treatments. According to an exemplary embodiment, as shown in FIG. 3, image guide 312 can be connected to video monitor 314. During the treatment, images relayed from the treatment site, i.e., at a distal end of image guide 312, are relayed to video monitor 314 for viewing by the doctor performing the treatment.

For insertion into the urethra, the distal ends of optical fiber 304, fluid channels 308 and 310 and fiber optic scope 312 are preferably contained within catheter structure 316. A variety of commonly available catheter and endoscope structures are suitable for use with device 300, and include, but are not limited to, those available from the Richard Wolf Medical Instruments Corporation. As described above, the catheter structure having the image guide therein, can first be inserted into the urethra and positioned just short of the treatment site, followed by the optical fiber and associated fluid channels.

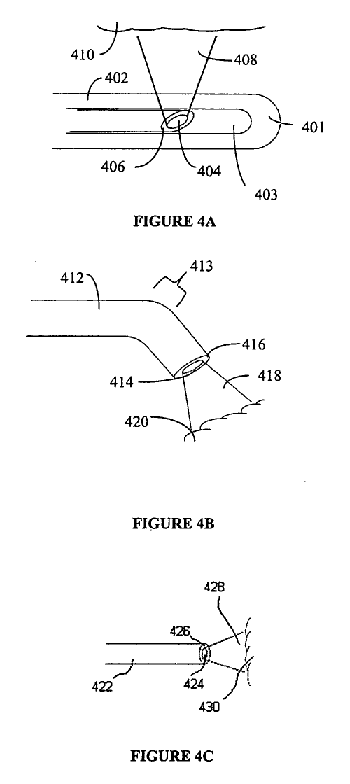

As described above, the optical fiber, i.e., optical fiber 304, is capable of distributing high laser power obliquely/perpendicularly to a longitudinal axis of the fiber. As shown in FIGS. 4A, 4B and 4C this can be achieved in a number of different ways. FIG. 4A is a diagram illustrating optical fiber 402 having a side-firing tip. Core 404/cladding 406 have a slanted surface formed thereon. The side-firing tip of optical fiber 402, with a near critical total reflectance angle, produces conical beam 408, to treat surface 410. The distal end of the optical fiber with slanted surface is covered with a quartz cap 401 having an air gap 403 facilitating side firing. FIG. 4B is a diagram illustrating optical fiber 412, with core 414/cladding 416, having bent tip 413 to direct exiting beam toward the treatment site. Namely, the tip of optical fiber 412 is bent at an angle of up to about 90 degrees, so as to direct beam 418 towards surface 420. The bent angle can be set into the distal fiber tip as disclosed, for example, in U.S. Pat. No. 5,553,177, which is hereby expressly incorporated by reference as part of the present disclosure. Alternatively, a means to tilt/bend the distal end, after placement at the treatment site, is present within the delivery system to position the fiber. FIG. 4C is a diagram illustrating an optical fiber 422 with core 424/cladding 426, having a straight tip to direct exiting beam 428 toward treatment site 430. As may be recognized by those of ordinary skill in the pertinent art based on the teachings herein, the optical fibers and other components of the device may take any of numerous different configurations that are currently known, or that later become known, including, for example, any of the configurations disclosed in commonly-owned, co-pending U.S. patent application Ser. No. 11/592,598, filed Nov. 3, 2006, entitled “Side Fire Optical Fiber For High Power Applications”, and U.S. patent application Ser. No. 60/962,106, filed Jul. 26, 2007, entitled “Adapter For Endoscopes”, each of which is hereby expressly incorporated by reference as part of the present disclosure.

Having described preferred embodiments of the invention with reference to the accompanying drawings, it is to be understood that the invention is not limited to the precise embodiments, and that various changes and modifications may be effected therein by one skilled in the art without departing from the scope of the invention as defined in the appended claims.

What is claimed is:

1. A method for treating benign prostatic hyperplasia while a patient is in a doctor's office or clinic, comprising the steps of:
i. identifying a portion of the patient's prostate to be removed;
ii. administering a local anesthetic along a treatment path extending along at least a portion of the patient's urethra;
iii. introducing an optical fiber along the treatment path until a distal end of the optical fiber is adjacent to a treatment site on the identified portion of the patient's prostate to be removed;
iv. transmitting laser radiation through the distal end of the optical fiber onto the treatment site at a power of at least about 100 Watts and at a wavelength within the range of at least one of (i) about 960 nm to about 1000 nm and (ii) about 1400 nm to about 1520 nm and, in turn, ablating and removing substantially entirely the identified portion of the patient's prostate; and
v. performing the foregoing steps while the patient is located in the doctor's office or clinic.

2. The method of claim 1, wherein the transmitting step comprises transmitting laser radiation onto the treatment site at a wavelength that is absorbed in blood and substantially preventing bleeding at the treatment site, and transmitting radiation onto the treatment site at a wavelength that is absorbed in water and at a power density sufficient to ablate and substantially entirely remove the identified portion of the patient's prostate.

3. The method of claim 1, further comprising the step of providing a high power laser radiation source of at least about 100 Watts optically coupled to an optical fiber having a proximal end and a distal end, wherein the optical fiber is capable of handling high laser power and distributing it at least one of obliquely and perpendicularly to a longitudinal axis of the fiber.

4. The method of claim 1, further comprising the step of introducing a scope including an image guide into the patient's urethra, and advancing the scope/image guide along the treatment path just short of the treatment site.

5. The method of claim 1, further comprising the step of providing an ice cold irrigating solution to the treatment site.

6. The method of claim 5, further comprising providing the ice cold irrigating solution to the treatment site at a temperature within the range of about 0° C. to about 5° C.

7. The method of claim 1, wherein the administering step comprises administering a saline solution of lidocaine.

8. The method of claim 1, wherein the administering step comprises:
(a) transrectal ultrasound and injection of lidocaine into a periprostatic space;
(b) insertion of a lidocaine jelly into the urethra; and
(c) intraprostatic injection of lidocaine.

9. The method of claim 1, further comprising performing steps i though v within a time period of less than about 1 hour.

10. The method of claim 8, further comprising (i) performing steps i through v within a time period of less than about 45 minutes, and (ii) performing step iv within a time period with the range of about 10 minutes to about 25 minutes.

11. The method of claim 1, further comprising maintaining the patient conscious throughout steps i through v.

12. The method of claim 1, wherein the transmitting step includes transmitting pulsed radiation.

13. The method of claim 12, further comprising pulsing the radiation on for a duration of not more than about 0.2 second and off for a duration of not more than about 0.01 second.

14. The method of claim 1, wherein the transmitting step includes transmitting CW radiation.

15. The method of claim 1, wherein the transmitting step comprises pulsing the radiation on and off for durations defined by the user.

16. The method of claim 1, further comprising the step of administering at least one antibacterial agent to the treatment site.

17. The method of claim 1, wherein the transmitting step comprises transmitting laser radiation at a wavelength that is within the range of about 960 nm to about 1000 mm and substantially simultaneously transmitting a radiation that is within the wavelength of about 1400 to about 1520 nm.

18. The method of claim 1, further comprising the step of increasing the power level to reduce bleeding at the treatment site.

19. An apparatus for treating benign prostatic hyperplasia while a patient is in a doctor's office or clinic, comprising:
i. first means for identifying a portion of the patient's prostate to be removed;
ii. second means for locally anesthetizing a treatment path extending along at least a portion of the patient's urethra;
iii. third means for introducing along the treatment path until a distal end thereof is adjacent to a treatment site on the identified portion of the patient's prostate to be removed;
iv. fourth means for transmitting laser radiation through the distal end of the third means onto the treatment site at a power of at least about 100 Watts and at a wavelength within the range of at least one of (i) about 960 nm to about 1000 nm and (ii) about 1400 nm to about 1520 nm and, in turn, ablating and removing substantially entirely the identified portion of the patient's prostate; and
v. wherein the first through fourth means each are configured for use within the doctor's office or clinic.

20. The apparatus of claim 19, wherein the first means is an imaging device, the second means is a local anesthetic, the third means is a fiber optic line, and the fourth means is a laser generator.

21. The apparatus of claim 19, further comprising means for cooling the treatment site to a temperature within the range of about 0° C. to about 5° C.

22. The apparatus of claim 21, wherein the means for cooling is an ice cold irrigating solution.

23. An apparatus for treating benign prostatic hyperplasia while a patient is in a doctor's office or clinic, comprising:
i. at least one laser radiation source located within the doctor's office or clinic that emits laser radiation at a power level of at least about 100 Watts and at wavelengths within the range of at least one of (i) about 960 nm to about 1000 nm and (ii) about 1400 to about 1520 nm;
ii. at least one optical fiber optically coupled to the laser radiation source, wherein the optical fiber defines a proximal end and a distal end that delivers the laser radiation at a power level of at least about 100 Watts;
iii. an image guide having a proximal end and a distal end, wherein the distal end of the image guide is associated with the distal end of the optical fiber and the proximal end of the image guide is optically connected to a monitor; and
iv. one or more fluid channels each having a proximal end and a distal end, wherein the distal end of each fluid channel is associated with the distal end of the optical fiber and the proximal end of at least one of the fluid channels is coupled in fluid communication with a fluid source.

24. The apparatus of claim 23, wherein the apparatus is mobile.

25. The apparatus of claim 23, wherein the apparatus is capable of running off at least one of a 110 volt alternating current power source and a 220 volt alternating current power source.

26. The apparatus of claim 23, wherein the optical fiber is capable of distributing the laser power at least one of obliquely and perpendicularly to a longitudinal axis of the optical fiber.

27. The apparatus of claim 26, wherein the optical fiber is one of a side firing fiber, a bent fiber and a straight fiber.
